(19) United States
(12) Patent Application Publication
(10) Pub. No.: US 2023/0031792 A1
(43) Pub. Date: Feb. 2, 2023

(54) Design Method of Oncological Computerized Physician Order Entry System with Intelligent Clinical Decision Recommendation Function

(71) Applicant: Zhejiang Haixinzhihui Technology Co., Ltd., Hangzhou City (CN)

(72) Inventors: LI Yingyun, Hangzhou City (CN); ZHU Dan, Hangzhou City (CN); JI Xiaosen, Hangzhou City (CN)

(73) Assignee: Zhejiang Haixinzhihui Technology Co., Ltd., Hangzhou City (CN)

(21) Appl. No.: 17/505,654

(22) Filed: Oct. 20, 2021

(30) Foreign Application Priority Data
Aug. 2, 2021 (CN) 2021108792757

(51) Int. Cl. G16H 50/20; G06F 16/21; G06N 5/02; G16H 10/60

(52) U.S. Cl. CPC G16H 50/20; G06F 16/21; G06N 5/022; G16H 10/60

(57) ABSTRACT

This invention disclosed a design method of an oncological computerized physician order entry system with an intelligent clinical decision recommendation function. The design method comprises the following steps: establish an intelligent decision-making system; integrate different data into a database through CDSS, propose individual recommendations and help clinicians optimize treatment plans according to the differences in drug efficacy, product accessibility, adverse reactions, patients' economic status and health care coverage; establish an intelligent decision-making system to provide individual recommendations and help clinicians optimize treatment plans; establish a knowledge database to perform standard integration and comprehensive utilization of tumor clinical medical data using such techniques as natural language normalization technique, structured technique and master patient indexing technique; a program is used to retrieve literature and automatically retrieve relevant clinical studies according to the different stages of tumor patients.

### TECHNICAL FIELD

This invention involves medical technology, in particular, a design method of oncological computerized physician order entry system with intelligent clinical decision recommendation function.

### BACKGROUND TECHNIQUE

The computerized physician order entry (CPOE) system is an important part of the hospital information system. Medical staff can diagnose and treat the patients based on physician orders during the process of medical diagnosis and treatment. The CPOE system is used to enter and manage physician orders, making physician orders clearer, more standard and normative. Compared with traditional hand-written prescriptions, it can greatly reduce human errors in writing and transcribing the prescriptions. At present, most CPOEs are general versions suitable for all departments of the hospitals, and more suitable for business and process management in the medical process.

The standard diagnosis and treatment plan for specific diseases is not reflected in the CPOE. If we rely only on individual physician's knowledge and experience, this will inevitably result in omissions or even errors, the service gaps between different medical institutions and different medical staff, and the failure to guarantee patients' safety and satisfaction. In this regard, we proposed a design method for oncological computerized physician order entry system with intelligent clinical decision recommendation function.

### CONTENT OF INVENTION

This invention aims to provide a design method of oncological computerized physician order entry system with intelligent clinical decision recommendation function to solve the technical problems in the background technology.

In order to achieve the above purpose, this invention provides the following technical solutions:

A design method of oncological computerized physician order entry with intelligent clinical decision recommendation function includes the following steps:

S1. Establish an intelligent decision-making system;

Integrate different data into the database through CDSS, propose individual recommendations and help clinicians optimize treatment plans according to the differences in drug efficacy, product accessibility, adverse reactions, patients' economic status and health care coverage;

S2. Establish a knowledge database;

The CSCO big data platform provides a structured design for the unstructured tumor clinic-pathological data described in natural language by defining and establishing a tumor structured standard data set; besides, the clinical information distributed in different information systems of different hospitals is collected, cleaned, and integrated for further standard integration and comprehensive utilization of tumor clinical medical data using such techniques as natural language normalization technique, structured technique and master patient indexing technique; a program is used to retrieve literature and automatically retrieve relevant clinical studies according to the different stages of tumor patients, then extract and build models for the individual characteristics of patients (such as different TNM stages, molecular typing, medication history and adverse reactions) in the literature; on the basis of summarizing the literature, the expert group in invited to make supplementary comments and improvement, and finally establish a knowledge graph model for tumor diagnosis and treatment corresponding to different types of patients;

S3. Construct a knowledge graph;

After data collection, technical means, together with man-machine interaction, should be used to integrate data, and map abstract data to graphic elements, helping users effectively perceive and analyze data, integrate knowledge resources and improve knowledge service capacity;

S4. Establish man-machine communication;

The man-machine interface plays an important role in interaction, and a reasonable communication interface is helpful for the promotion and application of the system, thereby helping improve the rationality of treatment;

S5. Embed the knowledge base into the computerized physician order entry to realize the intelligent recommendation function.

Preferably, this invention takes tumor diagnosis and treatment as the time axis through CSCO AI system, and the system firstly determines such critical information as basic information, current stage and previous treatment of the patients after entering the patients' age, TNM staging, molecular typing and other important information, and then gives the subsequent treatment method, treatment path, and specific treatment recommendations for the doctor's reference based on the patients' actual conditions.

Preferably, after the physician has selected the specific plan so recommended, a link to the knowledge graph centered with this treatment plan can be provided, and clinical evidences, references, usage & dosage of related drugs, drug name, side effects and other entity relationship of CSCO guidelines are presented; it can also show the diagnosis and treatment opinions of domestic experts synchronously for the exchange of views.

Preferably, the CSCO AI can integrate health care coverage, clinical studies and other data to make it more in line with domestic conditions, ensure that the users can obtain more information through one click, and improve the efficiency of clinicians.

Preferably, the application and promotion of the CDSS require clinical verification and user feedback, so as to optimize the system, and the verification methods for the CSCO AI system include the stages of identifying effective information, decision making, verifying decisions and marketing.

Preferably, the clinical study aims to identify the integrity of effective information: CSCO AI is an intelligent system built by technicians through technical means under the guidance of oncology experts; due to the difference in the professional background of the two, there is a gap in understanding the critical information of diagnosis and treatment; hence, clinical study aims to determine the critical information required for decision-making by the system through testing and learning the cases at different stages, set the information priority, and position the system to give a decision boundary, laying a foundation for the accordance rate of the subsequent judgments, decisions and guidelines.

Preferably, the research method is as follows: screen the patients with advanced recurrent metastatic tumors after operation based on real tumor data in the CSCO database, make intelligent decisions for each patient in three stages of initial treatment, post-operative adjuvant treatment and first-line treatment, analyze the probability for CSCO AI to successfully make an intelligent decision, and understand the priority of critical information.

Preferably, the research result is as follows: 200 patients with recurrent metastatic tumors are screened from the CSCO database, and the CSCO AI system can provide intelligent decision solutions in the stages of initial treatment, postoperative adjuvant treatment and first-line salvage treatment, in order to achieve the goals of clinical study.

Compared with existing technologies, this invention has the following advantages:

1. It can provide individual recommendations and help the clinicians optimize treatment plans by establishing an intelligent decision-making system, and using such techniques as natural language normalization technique, structured technique and master patient indexing technique to establishing a knowledge base to perform further standard integration and comprehensive utilization of tumor clinical medical data; a program is used to retrieve literature and automatically retrieve relevant clinical studies according to the different stages of tumor patients, then extract and build model for the individual characteristics of patients (such as different TNM stages, molecular typing, medication history and adverse reactions) in the literature; on the basis of summarizing the literature, the expert group is invited to make supplementary comments and improvement, finally establish a knowledge graph model for tumor diagnosis and treatment corresponding to different types of patients, and provide assistance to the clinicians according to the literature, plan, case report form, treatment experience, etc.;

2. It transforms the content of the international standard clinical guidelines into practical clinical diagnosis and treatment steps, and embeds them into the CPOE system used by medical staff in daily work to standardize the medical staff's diagnosis and treatment behaviors. For primary medical institutions, it is also an important means to improve their medical service level. This invention does not impose restrictions on the diagnosis and treatment behaviors of medical staff. The intelligent clinical decision recommendation is a supplement to the individual knowledge and experience of medical staff and aims to check the shortcomings and fill in the omissions, thus effectively avoiding human-made errors and safeguarding the patients' safety.

### DRAWINGS

FIG. 1 is a schematic diagram of the overall process of this invention.

### EMBODIMENTS

The technical solutions in the embodiments of this invention are clearly and fully described in accordance with the drawings in the embodiments. Obviously, the described embodiments are only some embodiments of this invention. Based on the embodiments of this invention, all other embodiments obtained by those skilled in the art without creative work shall fall within the protection scope of this invention.

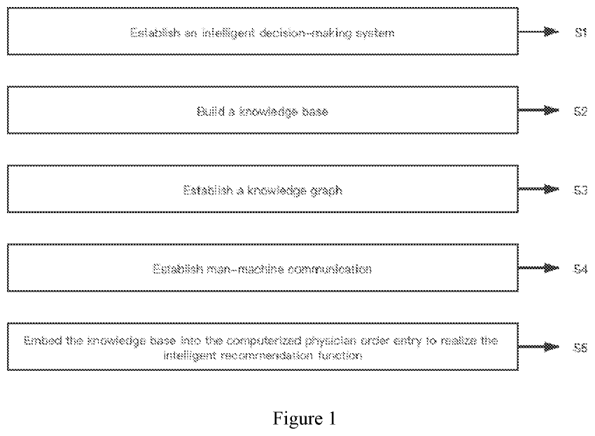

Please refer to FIG. 1. This invention provides a technical solution:

A design method of oncological computerized physician order entry with intelligent clinical decision recommendation function includes the following steps:

S1. Establish an intelligent decision-making system;

Integrate different data into the database through CDSS, propose individual recommendations and help clinicians optimize treatment plans according to the differences in drug efficacy, product accessibility, adverse reactions, patients' economic status and health care coverage;

S2. Establish a knowledge database;

The CSCO big data platform provides a structured design for the unstructured tumor clinic-pathological data described in natural language by defining and establishing a tumor structured standard data set; besides, the clinical information distributed in different information systems of different hospitals is collected, cleaned, and integrated, for further standard integration and comprehensive utilization of tumor clinical medical data using such techniques as natural language normalization technique, structured technique and master patient indexing technique; a program is used to retrieve literature and automatically retrieve relevant clinical studies according to the different stages of tumor patients, then extract and build models for the individual characteristics of patients (such as different TNM stages, molecular typing, medication history and adverse reactions) in the literature; on the basis of summarizing the literature, the expert group is invited to make supplement and improvement, and finally establish a knowledge graph model for tumor diagnosis and treatment corresponding to different types of patients;

S3. Construct a knowledge graph;

After data collection, technical means, together with man-machine interaction, should be used to integrate data, and map abstract data to graphic elements, helping users effectively perceive and analyze data, integrate knowledge resources and improve knowledge service capacity;

S4. Establish man-machine communication;

The man-machine interface plays an important role in interaction, and a reasonable communication interface is helpful for the promotion and application of the system, thereby helping improve the rationality of treatment;

S5. Embed the knowledge base into the computerized physician order entry to realize the intelligent recommendation function.

Please refer to FIG. 1. The present invention takes tumor diagnosis and treatment as the time axis through CSCO AI system, and the system firstly determines such critical information as basic information, current stage and previous treatment of the patients after entering the patients' age, TNM staging, molecular typing and other important information, and then gives the subsequent treatment method, treatment path, and specific treatment recommendations for the doctor's reference based on the patients' actual conditions;

Please refer to FIG. 1. After the physician has selected the specific plan so recommended, a link to the knowledge graph centered with this treatment plan can be provided, and clinical evidences, references, usage & dosage of related drugs, drug name, side effects and other entity relationship of CSCO guidelines are presented; it can also show the diagnosis and treatment opinions of domestic experts synchronously for the exchange of views.

Please refer to FIG. 1. The CSCO AI can integrate health care coverage, clinical studies and other data to make it more in line with domestic conditions, ensure that the users can obtain more information through one click, and improve the efficiency of clinicians.

Please refer to FIG. 1. The application and promotion of the CDSS require clinical verification and user feedback, so as to optimize the system, and the verification methods for the CSCO AI system include the stages of identifying effective information, decision making, verifying decisions and marketing;

Please refer to FIG. 1. The clinical study aims to identify the integrity of effective information: CSCO AI is an intelligent system built by technicians through technical means under the guidance of oncology experts; due to the difference in the professional background of the two, there is a gap in understanding the critical information of diagnosis and treatment; hence, clinical study aims to determine the critical information required for decision making by the system through testing and learning the cases at different stages, set the information priority, and position the system to give a decision boundary, laying a foundation for the accordance rate of the subsequent judgments, decisions and guidelines.

Please refer to FIG. 1. The research method is as follows: screen the patients with advanced recurrent metastatic tumors after operation based on real tumor data in the CSCO database, make intelligent decisions for each patient in three stages of initial treatment, post-operative adjuvant treatment and first-line treatment, analyze the probability for CSCO AI to successfully make an intelligent decision, and understand the priority of critical information.

Please refer to FIG. 1. The research result is as follows: 200 patients with recurrent metastatic tumors are screened from the CSCO database, and the CSCO AI system can provide intelligent decision solutions in the stages of initial treatment, postoperative adjuvant treatment and first-line salvage treatment, in order to achieve the goals of clinical study;

During use, this invention can provide individual recommendations and help the clinicians optimize treatment plans by establishing an intelligent decision-making system, and use such techniques as natural language normalization technique, structured technique and master patient indexing technique to establish a knowledge base for further standard integration and comprehensive utilization of tumor clinical medical data; a program is used to retrieve literature and automatically retrieve relevant clinical studies according to the different stages of tumor patients, then extract and build models for the individual characteristics of patients (such as different TNM stages, molecular typing, medication history and adverse reactions) in the literature; on the basis of summarizing the literature, the expert group is invited to make supplementary comments and improvement, finally establish a knowledge graph model for tumor diagnosis and treatment corresponding to different types of patients, and provide assistance to the clinicians according to the literature, plan, case report form, treatment experience, etc. This invention transforms the content of the international standard clinical guidelines into practical clinical diagnosis and treatment steps, and embeds them into the CPOE system used by medical staff in daily work to standardize the medical staff's diagnosis and treatment behaviors. For primary medical institutions, it is also an important means to improve their medical service level. This invention does not impose restrictions on the diagnosis and treatment behaviors of medical staff. The intelligent clinical decision recommendation is a supplement to the knowledge and experience of medical staff and aims to check the shortcomings and fill in the omissions, thus effectively avoiding human-made errors and safeguarding the patients' safety.

Although the embodiments of the present invention have been illustrated and described, it will be apparent to those skilled in this field that various changes, modifications, substitutions and variations can be made to these embodiments without departing from the principle and spirit of this invention, and the scope of this invention is defined by the appended claims and their equivalents.

We claim:

1. A design method of oncological computerized physician order entry system with intelligent clinical decision recommendation function is characterized in the following steps included:
S1. Establish an intelligent decision-making system;
Integrate different data into the database through CDSS, propose individual recommendations and help clinicians optimize treatment plans according to the differences in drug efficacy, product accessibility, adverse reactions, patients' economic status and health care coverage;
S2. Establish a knowledge database;
The CSCO big data platform provides a structured design for the unstructured tumor clinic-pathological data described in natural language by defining and establishing a tumor structured standard data set; besides, the clinical information distributed in different information systems of different hospitals is collected, cleaned, and integrated for further standard integration and comprehensive utilization using such techniques as natural language normalization technique, structured technique and master patient indexing technique; a program is used to retrieve literature and automatically retrieve relevant clinical studies according to the different stages of tumor patients, then extract and build models for the individual characteristics of patients (such as different TNM stages, molecular typing, medication history and adverse reactions) in the literature; on the basis of summarizing the literature, the expert group is invited to make supplementary comments and improvement, and finally establish a knowledge graph model for tumor diagnosis and treatment corresponding to different types of patients;
S3. Construct a knowledge graph;
After data collection, technical means, together with man-machine interaction, should be used to integrate data, and map abstract data to graphic elements, helping users effectively perceive and analyze data, integrate knowledge resources and improve knowledge service capacity;
S4. Establish man-machine communication;
The man-machine interface plays an important role in interaction, and a reasonable communication interface is helpful for the promotion and application of the system, thereby helping improve the rationality of treatment;
S5. Embed the knowledge base into the computerized physician order entry system to realize the intelligent recommendation function.

2. The feature of the design method of oncological computerized physician order entry system with intelligent clinical decision recommendation function according to claim 1 is: this invention takes tumor diagnosis and treatment as the time axis through CSCO AI system, and the system firstly determines such critical information as basic information, current stage and previous treatment of the patients after entering the patients' age, TNM staging, molecular typing and other important information, and then gives the subsequent treatment method, treatment path, and specific treatment recommendations for the physician's reference based on the patients' actual conditions.

3. The feature of the design method of oncological computerized physician order entry system with intelligent clinical decision recommendation function according to claim 2 is: after the physician has selected the specific plan so recommended, a link to the knowledge graph centered with this treatment plan can be provided, and clinical evidence, references, usage & dosage of related drugs, drug name, side effects and other entity-relationship of CSCO guidelines are presented; it can also show the diagnosis and treatment opinions of domestic experts synchronously for the exchange of views.

4. The feature of the design method of oncological computerized physician order entry system with intelligent clinical decision recommendation function according to claim 2 is: the CSCO AI can integrate health care coverage, clinical studies and other data to make it more in line with domestic conditions, ensure that the users can obtain more information through one click, and improve the efficiency of the clinicians.

5. The feature of the design method of oncological computerized physician order entry system with intelligent clinical decision recommendation function according to claim 1 is: the application and promotion of the CDSS require clinical verification and user feedback, so as to optimize the system, and the verification methods for the CSCO AI system include the stages of identifying effective information, decision-making, verifying decisions and marketing.

6. The feature of the design method of oncological computerized physician order entry system with intelligent clinical decision recommendation function according to claim 5 is: the clinical study aims to identify the integrity of effective information: CSCO AI is an intelligent system built by technicians through technical means under the guidance of oncology experts; due to the difference in the professional background of the two, there is a gap in understanding the critical information of diagnosis and treatment; hence, the clinical study aims to determine the critical information required for decision-making by the system through testing and learning the cases at different stages, set the information priority, and position the system to give a decision boundary, laying foundation for the accordance rate of the subsequent judgments, decisions and guidelines.

7. The feature of the design method of oncological computerized physician order entry system with intelligent clinical decision recommendation function according to claim 1 is: the research method is as follows: screen patients with advanced recurrent metastatic tumors after operation based on real tumor data in the CSCO database, make intelligent decisions for each patient in three stages of initial treatment, postoperative adjuvant treatment and first-line treatment, analyze the probability for CSCO AI to successfully make an intelligent decision, and understand the priority of critical information.

8. The feature of the design method of oncological computerized physician order entry system with intelligent clinical decision recommendation function according to claim 1 is: the research result is as follows: 200 patients with recurrent metastatic tumors are screened from the CSCO database, and the CSCO AI system can provide intelligent decision solutions in the stages of initial treatment, postoperative adjuvant treatment and first-line salvage treatment, in order to achieve the goals of the clinical study.
